(19) United States
(12) Patent Application Publication
(10) Pub. No.: US 2018/0104190 A1
(43) Pub. Date: Apr. 19, 2018

(54) ABUSE DETERRENT PHARMACEUTICAL COMPOSITIONS

(71) Applicant: KVK-Tech, Inc., Newtown, PA (US)

(72) Inventors: Murty Vepuri, Newtown, PA (US); Jayendra Patel, Bensalem, PA (US)

(21) Appl. No.: 15/845,819

(22) Filed: Dec. 18, 2017

Related U.S. Application Data
(63) Continuation-in-part of application No. 15/580,524.
(60) Provisional application No. 62/173,183, filed on Jun. 9, 2015.

(51) Int. Cl. A61K 9/20; A61K 9/00; A61K 31/485; A61K 9/28; A61P 25/04

(52) U.S. Cl. CPC A61K 9/205; A61K 9/0053; A61K 31/485; A61P 25/04; A61K 9/2027; A61K 9/2013; A61K 9/284; A61K 9/2031

(57) ABSTRACT

Abuse deterrent pharmaceutical compositions, processes for their preparation and methods of use thereof are described. An exemplary composition is a solid oral pharmaceutical composition containing an active agent, a gelling agent in an amount of between about 0.7 and about 1.5% of the weight of the composition, and a channeling agent in an amount of at least about 40% of the weight of the composition. In one embodiment, the active agent is an opioid such as oxycodone. In one embodiment, the gelling agent is xanthan gum. The gelling agent deters the extractability of the drug from the composition. In another embodiment, the channeling agent is crospovidone. The channeling agent allows the immediate release of the active agent from the composition in the presence of the gelling agent. In a preferred embodiment, crospovidone is in an amount about 53.3% of the weight of the composition.

### CROSS-REFERENCE TO RELATED APPLICATIONS

This application is a continuation-in-part of U.S. Ser. No. 15/580,524, filed Dec. 7, 2017, which is a 371 application of PCT/US2016/036470, filed Jun. 8, 2016, which claims priority to and benefit of U.S. Provisional Application No. 62/173,183, filed Jun. 9, 2015, the disclosures of which are hereby incorporated herein by reference in their entirety.

### FIELD OF THE INVENTION

The present invention relates to abuse deterrent pharmaceutical compositions, processes for their preparation and methods of use thereof.

### BACKGROUND OF THE INVENTION

Many psychoactive or analgesic pharmaceutical drugs have a significant ability to cause euphoria or pleasurable effects and are thereby at risk for abuse. Some of the commonly abused drugs are the opioids, sedatives, stimulants and hypnotics. In many instances, the solid dosage forms containing abused drugs are crushed, melted, dissolved or altered; and they are then inhaled, snorted, or injected in a manner that is inconsistent with their safe usage. Tampering of solid dosage forms in particular will rapidly deliver a massive dose and produce a variety of serious and life threatening side effects, including respiratory depression and failure, sedation, cardiovascular collapse, coma and death.

One pharmaceutical drug class that is particularly tampered with is the opioids. A particular dose of an opioid agonist may be more potent when administered by intranasal (e.g. snorting) or parenteral (e.g. intravenous) routes relative to the same dose administered orally. These methods result in the rapid bioavailability of relatively high doses of drug, giving the abuser a “high”.

Various technologies to prevent tampering or drug-abuse have been developed but each has met with limited success. One approach is to include high molecular weight polymers that confer plasticity to the dosage form, rendering them difficult to crush and pulverize into a powder. Another way of substantially reducing or even eliminating this potential for drug abuse is to suppress or inhibit the extraction of the active ingredient from the tablet by the use of gelling agents, thus preventing the drug from being abused parenterally. However, the high molecular weight polymers and gelling agents retard the release of the active ingredient from the dosage forms, making them typically unsuitable for immediate release formulations.

Thus, there is a need for oral solid pharmaceutical compositions that provide immediate release of an active agent susceptible to abuse yet are resistant to misuse.

It is therefore an object of the present invention to provide an oral solid pharmaceutical composition that reduces the potential for improper administration or use of drugs but which, when administered as directed, is capable of delivering a therapeutically effective dose.

It is yet another object of the present invention to provide a method of manufacturing an oral solid pharmaceutical composition.

It is a further object of the present invention to provide a method of treating pain by administering an oral solid pharmaceutical composition to a patient in need thereof.

### SUMMARY OF THE INVENTION

Abuse deterrent pharmaceutical compositions and methods of making and using are described herein. An exemplary composition is a solid oral pharmaceutical composition containing an active agent, a gelling agent in an amount of between about 0.7 and about 1.5% of the weight of the composition, and a channeling agent in an amount of at least about 40% of the weight of the composition. In one embodiment, the active agent is an opioid such as oxycodone. In one embodiment, the gelling agent is xanthan gum. The gelling agent deters the extractability of the drug from the composition. In another embodiment, the channeling agent is crospovidone. The channeling agent allows the immediate release of the active agent from the composition in the presence of the gelling agent. In a preferred embodiment, crospovidone is in an amount about 53.3% of the weight of the composition.

In preferred embodiments, the composition contains polyethylene oxide (also in general referred as polyglycols) a cationic polymer amino alkyl methacrylate copolymer, a lubricant, and a film coating. Polygylcols plasticity to the solid oral pharmaceutical composition, rendering them difficult to crush and pulverize into a powder. In preferred embodiments, the polyglycol is polyethylene oxide with an average molecular weight of between about 900,000 daltons and about 7,000,000 daltons.

The composition can be in the form of an immediate release tablet formed of a polymer matrix containing a cationic polymer. In one embodiment, the cationic polymer is a methacrylic acid derivative with a amino alkyl methacrylate group. In a preferred embodiment, the cationic polymer is poly(butyl methacrylate-co-(2-dimethylaminoethyl)methacrylate-co-methyl methacrylate) 1:2:1. Lubricants prevent ingredients of the composition from clumping together. In a preferred embodiment, the lubricant is magnesium stearate. In preferred embodiments, the film coating agent contains polyvinylalcohol.

The pharmaceutical composition can be manufactured by forming a mixture containing an active agent, a gelling agent and a channeling agent; and forming a solid dosage unit from the mixture.

The pharmaceutical composition is administered to or prevent pain in a patient in need thereof.

### BRIEF DESCRIPTION OF THE DRAWING

FIG. 1 displays the in vitro dissolution profiles in water of Composition 1 (example 1) and oxycodone hydrochloride tablets 15 mg immediate release commercially available.

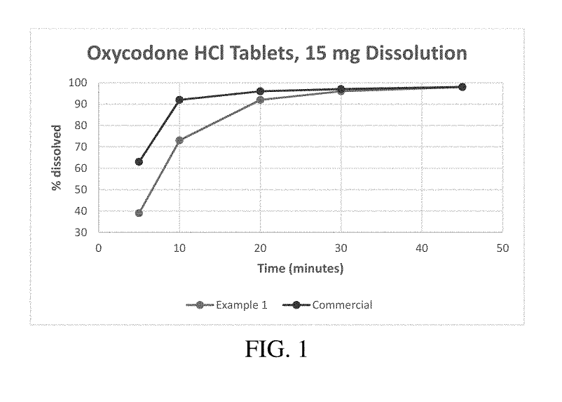

### DETAILED DESCRIPTION OF THE INVENTION

### I. Definitions

“Abuse deterrent”, as used herein, refers to dosage forms which at least provide resistance to crushing or resistance to aqueous and alcohol extractions, preferably both.

As used herein, the terms “active agent”, “pharmaceutical agent”, and “drug” refer to any material that is intended to produce a therapeutic, prophylactic, or other intended effect. These terms with respect to specific agents include all pharmaceutically active agents, all pharmaceutically acceptable salts thereof, hydrates and solvates thereof, and mixtures thereof. used herein, the term “therapeutically effective” refers to the amount of active agent needed to produce a desired therapeutic result.

The term “immediate release”, as used herein, refers to an average release of at least 70% (q), as defined by USP, of the labeled amount of active agent within 45 minutes.

“Gelling agent”, as used herein, refers to a material which forms a gel by the action of an aqueous medium, such as water or an aqueous solution of an organic acid (e.g. aqueous citric or acetic acid), a base (e.g. sodium bicarbonate or sodium tetraborate solution) or alcohol (e.g. an aqueous lower alkanol such as aqueous ethanol or isopropanol), or combinations thereof.

The term “channeling agent”, as used herein, includes water-soluble excipients, which can be solubilized in water or gastrointestinal fluid, thus forming channels through which the water or the gastrointestinal fluid enters the composition. This action aids in improving dissolution.

As used herein, “UPLC” refers to ultra-pressure liquid chromatography.

### II. Compositions

A. Active Agents

Active agents include, but are not limited to, opioids, depressants, stimulants, anti-anxiolytics (e.g., benzodiazepines), sedatives, hypnotics, stimulants, and cannabinoids, among others.

Opioids can be, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, piminodine, piritramide, propheptazine, promedol, properidine, propoxyphene, sufentanil, tilidine, and tramadol or pharmaceutically acceptable salts thereof. In one embodiment, the opioid is codeine, hydrocodone, hydromorphone, morphine, oxycodone, oxymorphone or tramadol. Preferably, the opioid is oxycodone.

Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, and solvates and mixtures thereof. Benzodiazepine antagonists that can be used include, but are not limited to, flumazenil as well as pharmaceutically acceptable salts, hydrates, and solvates.

Barbiturates are sedative-hypnotic drugs derived from barbituric acid (2,4,6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital and as well as pharmaceutically acceptable salts, hydrates, and solvates mixtures thereof.

Stimulants refer to drugs that stimulate the central nervous system. Stimulants include, but are not limited to, amphetamines, such as amphetamine, dextroamphetamine resin complex, dextroamphetamine, methamphetamine, methylphenidate as well as pharmaceutically acceptable salts, hydrates, and solvates and mixtures thereof.

Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, salt, dicyclohexylamine salt, and N,N′-dibenzylethylenediamine salt.

### B. Gelling Agents

The gelling agent imparts a gel-like quality to the dosage form in the presence of a liquid (e.g., an extracting solvent or within the mucosa) to hinder the ability to inject or inhale the active agent. Gelling agent include, but are not limited to, starch and starch derivatives, cellulose derivatives (e.g., microcrystalline cellulose, sodium carboxymethyl cellulose, methylcellulose, ethyl cellulose, hydroxyethyl cellulose, hydroxypropyl cellulose, and hydroxypropyl methylcellulose), attapulgites, bentonites, dextrins, alginates, carrageenan, gum tragacanth, gum acacia, guar gum, xanthan gum, pectin, gelatin, kaolin, lecithin, magnesium aluminum silicate, alginic acid derivates (e.g., sodium and calcium salts of alginic acid), chitin derivatives (e.g., chitosan), carbomers, carbopols, polycarbophils, polyvinylpyrrolidone, polyvinyl alcohol, silicon dioxide, and mixtures thereof. In preferred embodiments, the gelling agent is xanthan gum.

### C. Channeling Agents

Channeling agents can be used to further tailor the drug release from the compressed tablets. Channeling agents help in opening up the granules and or tablets in a specific media as desired Channeling agents include, but are not limited to, croscarmellose sodium, crospovidone and sodium starch glycolate, diluents such as lactose, mannitol, sodium chloride and talc. The channeling agent is leached out to form channels through the tablet at different rates depending on the solubilization rate of the channeling agent in the releasing medium. In preferred embodiments, the channeling agent is crospovidone.

### D. Excipients

In preferred embodiments, the pharmaceutical composition contains one or more additional pharmaceutically acceptable excipients. Non-limiting examples of suitable excipients include polyglycols, cationic polymers, lubricants, disintegrants, binders, fillers, diluents, antioxidants, chelating agents, flavoring agents, coloring agents, taste masking agents, and combinations thereof.

i. Polyglycols

Polygylcols confer plasticity to the solid oral pharmaceutical composition, rendering them difficult to crush and pulverize into a powder. In compositions containing a low molecular weight polyethylene oxide, the low molecular weight polyethylene oxide can, for example, have an average molecular weight between about 10,000 and about 750,000 daltons, between about 50,000 and about 500,000 daltons, or between about 75,000 and about 300,000 daltons.

In compositions containing a high molecular weight polyethylene oxide, the high molecular weight polyethylene oxide can, for example, have an average molecular weight between about 1,000,000 and about 10,000,000 daltons, between about 2,000,000 and about 8,000,000 daltons, or between about 4,000,000 and about 6,000,000 daltons.

In certain embodiments, the oral dosage form includes between about 2% (w/w) and about 20% (w/w) polyethylene oxide, between about 5% (w/w) and about 15% (w/w) polyethylene oxide, or between about 8% (w/w) and about 11% (w/w) polyethylene oxide and a combination of polyethylene oxide grades starting at 100,000 to 7,000,000 dalton units ranging between 2.0% to about 10% (w/w). In addition to, or in place of, polyethylene oxide, the oral dosage form can include a non-ionic triblock copolymer composed of a central hydrophobic chain of polyoxypropylene (poly(propylene oxide)) flanked by two hydrophilic chains of polyoxyethylene (poly (ethylene oxide)). These compounds are commercially available under the tradenames LUTROL® and POLOXAMER®.

ii. Cationic Polymers

The composition can be in the form of an immediate release tablet with a polymer matrix containing a cationic polymer such as methacrylic acid and derivatives thereof. Suitable methacrylic acid polymers include EUDRAGIT® EPO, EUDRAGIT® RLPO, EUDRAGIT® RSPO, and various derivatives of methacrylic acid esters and mixtures thereof.

iii. Lubricants

Lubricants prevent ingredients of the composition from clumping together. Non-limiting examples of suitable lubricants include magnesium stearate, calcium stearate, zinc stearate, hydrogenated vegetable oils, sterotex, polyoxyethylene monostearate, polyethylene glycol, sodium stearyl fumarate, sodium benzoate, sodium lauryl sulfate, magnesium lauryl sulfate, light mineral oil, and combinations thereof. In a preferred embodiment, the lubricant is magnesium stearate.

iv. Film Coating

The pharmaceutical composition can include a film coat. Typically, the film coat comprises a water-soluble polymer which does not affect the immediate release or tamper resistant properties of the composition. The film coating may provide moisture protection, enhanced appearance, increased mechanical integrity, improved swallowability, improved taste, and/or masking of odors.

Film coatings are well known in the art. In one embodiment, they are commercially available under the tradename OPADRY®. Typically, a film coating contains at least one water-soluble polymer and at least one plasticizer. Non-limiting examples of suitable polymers include celluloses such as hydroxypropyl methylcellulose, hydroxypropyl cellulose, hydroxypropyl ethylcellulose, ethylcellulose, methylcellulose, cellulose acetate phthalate, and microcrystalline cellulose, carrageenan, polyvinyl alcohol, anionic and cationic acrylic or methacrylic acid polymers and copolymers, copolymers of ethacrylate and methylmethacrylate, polyvinylacetate phthalate, and shellac.

Examples of suitable plasticizers for inclusion in the film coating include, triethyl citrate (TEC), acetyltriethyl citrate (ATEC), acetyl tri-n-butyl citrate (ATBC), dibutyl sebacate, diethyl phthalate, and triacetin. The film coating may contain a coloring agent, a filler, a flavoring agent, a taste-masking agent, a surfactant, an anti-tacking agent, and/or an anti-foaming agent. Suitable examples of these agents are well known in the art and/or are detailed above.

E. Dosage Forms

The physical form of the pharmaceutical compositions can vary. In general, the pharmaceutical compositions are solid dosage forms for oral administration. Suitable solid dosage forms include tablets, caplets, granules, pills, and capsules. Such dosage forms may be prepared using conventional methods known to those in the field of pharmaceutical formulation and described in the pertinent texts, e.g., in Gennaro, A. R., editor. “Remington: The Science & Practice of Pharmacy”, 21st ed., Williams & Williams, and in the “Physician's Desk Reference”, 2006, Thomson Healthcare.

In preferred embodiments, the solid dosage form is a tablet. Non-limiting types of tablets include coated tablets, uncoated tablets, compressed tablets, compacted tablets, molded tablets, layered tablets, bilayer tablets, extruded tablets, multiparticle tablets, monolithic tablets, and matrix tablets.

In general, the tablet has sufficient mechanical strength and/or resiliency that it is difficult to crush into a powder. The mechanical strength of the tablet may be quantified by its hardness or crushing strength, friability, and/or tensile strength. In preferred embodiments, the tablet may have a hardness or crushing strength of at least about 7 kilopond (kp). In various embodiments, the tablet may have a hardness or crushing strength that ranges from about 7 kp to about 10 kp, from about 10 kp to about 15 kp, from about 15 kp to about 20 kp, from about 20 kp to about 25 kp, or greater than 25 kp. In general, the tablet has a friability of no greater than about 1.0%, or more preferably no greater than about 0.5%. In certain embodiments, the tablet may have a friability of less than about 1.0%, less than about 0.5%, less than about 0.3%, less than about 0.2%, less than about 0.1%, less than about 0.05%, or less than about 0.01%.

In Vitro Release Properties of the Composition

The pharmaceutical composition is formulated such that the active agent in the composition is rapidly released, i.e., it is formulated as an immediate release composition. The in vitro dissolution of the active agent (or “API”) from the composition may be measured using an USP dissolution procedure for the tablet dosage form. For example, dissolution may be measured using an USP Type 2 paddle apparatus, at a paddle speed of 50 rpm or 100 rpm, and a constant temperature of 37±0.5° C. The dissolution may be performed in the presence of 500 mL, 900 mL, or 1,000 mL of a suitable dissolution medium (e.g., having a pH from 1.0 to 6.8). Non- limiting examples of suitable dissolution media include water, phosphate buffer (pH 6.8), acetate buffer (pH 4.5), and 0.1 HCl.

In some embodiments, the pharmaceutical composition may have an average release of about 50%, 55%, 60%, 65%, 70%, 75%, 80%, 85%, 90%, 95%, or 99% of the API within about 45 minutes. In other embodiments, the pharmaceutical composition may have an average release of about 50%, 55%, 60%, 65%, 70%, 75%, 80%, 85%, 90%, 95%, or 99% of the API within about 30 minutes.

### III. Methods of Manufacture

Solid dosage forms of the pharmaceutical compositions are prepared using process including the steps of (a) forming a mixture containing an active agent, a gelling agent and a channeling agent; (b) forming a solid dosage unit from the mixture; and (c) thermo-curing (heating) the solid dosage unit to form the solid dosage form.

The first step of the process includes forming a mixture containing the components of the pharmaceutical composition. The mixture components may be combined in any order or may be premixed in various combinations before being combined together. For example, the gelling agent and the channeling agent may be blended together before being combined with the rest of the components. Similarly, the active agent may be combined with some of the components before being combined with the rest of the components. Thus, a variety of ordered mixing schemes are possible. The mixture containing the components of the composition may be formed by mixing, roller mixing, drum mixing, shear mixing, dry blending, chopping, milling, granulating, dry granulating (e.g., slugging or roller compacting), wet granulating (e.g., fluid bed granulating, high shear granulating), and other mixing techniques known in the art.

The process further includes forming the mixture into a solid dosage unit. In exemplary embodiments, the solid dosage unit may be a tablet. The tablet may be a compression tablet, a molded tablet, a compacted tablet, or a pressed tablet. In an exemplary embodiment, the tablet may be formed by direct compression. The shape of the tablet may vary. Non-limiting tablet include round, oval, rectangular, and triangular. The size and mass of the tablet may vary. In various embodiments, the mass of the tablet can be about 100 mg to about 500 mg. In preferred embodiment, the mass of the tablet is about 150mg.

The final step of the process comprises heating (curing) the solid dosage unit to yield the solid dosage form. This heating step cures the solid dosage unit, wherein the cured solid dosage form may have improved properties or characteristics relative to an uncured solid dosage unit.

### IV. Methods of Use

The pharmaceutical composition is useful for reducing or preventing pain that is treatable with conventional compositions containing oxycodone. These include headache pain, pain associated with migraine, neuropathic pain such as diabetic neuropathy, HIV sensory neuropathy, post-herpetic neuralgia, post-thoracotomy pain, trigeminal neuralgia, radiculopathy, neuropathic pain associated with chemotherapy, reflex sympathetic dystrophy, back pain, peripheral neuropathy, entrapment neuropathy, phantom limb pain, and complex regional pain syndrome, dental pain, pain associated with a surgical procedure and or other medical intervention, bone cancer pain, joint pain associated with psoriatic arthritis, osteoarthritic pain, rheumatoid arthritic pain, juvenile chronic arthritis associated pain, juvenile idiopathic arthritis associated pain, Spondyloarthropathies (such as ankylosing spondylitis (Mb Bechterew) and reactive arthritis (Reiter's syndrome) associated pain), pain associated with psoriatic arthritis, gout pain, pain associated with pseudogout (pyrophosphate arthritis), pain associated with systemic lupus erythematosus (SLE), pain associated with systemic sclerosis (scleroderma), pain associated with Behcet's disease, pain associated with relapsing polychondritis, pain associated with adult Still's disease, pain associated with transient regional osteoporosis, pain associated with neuropathic arthropathy, pain associated with sarcoidosis, arthritic pain, rheumatic pain, joint pain, osteoarthritic joint pain, rheumatoid arthritic joint pain, juvenile chronic arthritis associated joint pain, juvenile idiopathic arthritis associated joint pain, Spondyloarthropathies (such as ankylosing spondylitis (Mb Bechterew) and reactive arthritis (Reiter's syndrome) associated joint pain), gout joint pain, joint pain associated with pseudogout (pyrophosphate arthritis), joint pain associated with systemic lupus erythematosus (SLE), joint pain associated with systemic sclerosis (scleroderma), joint pain associated with Behcet's disease, joint pain associated with relapsing polychondritis, joint pain associated with adult Still's disease, joint pain associated with transient regional osteoporosis, joint pain associated with neuropathic arthropathy, joint pain associated with sarcoidosis, arthritic joint pain, rheumatic joint pain, acute pain, acute joint pain, chronic pain, chronic joint pain, inflammatory pain, inflammatory joint pain, mechanical pain, mechanical joint pain, pain associated with the fibromyalgia syndrome (FMS), pain associated with polymyalgia rheumatica, monarticular joint pain, polyarticular joint pain, nociceptive pain, psychogenous pain, pain of unknown etiology, pain mediated by IL-6, IL-6 soluble receptor, or IL-6 receptor, pain associated with a surgical procedure in a patient with a clinical diagnosis of OA, pain like static allodynia, pain like dynamic allodynia, and/or pain associated with Crohn's disease.

The present invention will be further understood by reference to the following non-limiting examples.

### EXAMPLE 1

### Manufacture of Composition 1

Materials and Methods

TABLE 1
Composition 1 Ingredients

| Component | | | |
|---|---|---|---|
| No. | Ingredient | mg/tablet | % weight |
| 1 | Oxycodone HCl, USP | 15.00 | 10.00 |
| 2 | EUDRAGIT ® EPO | 35.00 | 23.33 |
| 3 | POLYOX ® WSR 303 Leo, NF | 4.00 | 2.67 |
| 4 | POLYOX ® WSR N 1105 Leo, | 12.00 | 8.00 |
| | NF | | |
| 5 | KOLLIDON ® 90 F (BASF) | 6.00 | 4.00 |
| 6 | Crospovidone, NF | 75.00 | 50.00 |
| 7 | Xanthan Gum, NF | 1.50 | 1.00 |
| 8 | Magnesium Stearate, NF | 1.50 | 1.00 |
| | Total Net | 150.00 | 100.00 |

Procedure: All components were sifted by screening each ingredient through a comil using 610 (equivalent to 30 m mesh) except for magnesium stearate. The screened materials were blended for 10 minutes. Magnesium stearate was added at the lubrication stage and the mixture was blended for an additional 5 minutes. The blend was compressed into a tablet dosage form. The tablets were film coated with OPADRY II® and cured.

For compositions containing 5 mg, 10 mg, 20 mg or 30 mg of oxycodone HCl in the tablet, the amount of EUDRAGIT® EPO used was adjusted to 45 mg, 40 mg, 30 mg or 20 mg, respectively. The amounts of components 3-8 remain the same for each tablet strength.

### Example 2

### Extraction of Oxycodone Using Solvents

Materials and Methods

Composition 1 and oxycodone hydrochloride tablets 15 mg (commercially available) were compared by extraction of the active with various solvents. The extraction solvents were the following: pH 1.2 buffer (USP Hydrochloric acid buffer); pH 6.8 Phosphate Buffer, pH 10.0 Buffer (USP Alkaline Borate Buffer); 40% ethanol; Ethanol; acetone; 1% acetic acid (aq); water; COCA-COLA®; and COCA-COLA®+40% Ethanol.

Procedure: Five tablets were crushed into a powder using a mortar and pestle. The powder was transferred to a beaker. 15 mL of the extraction solvent was added to the beaker and stirred. After 10 minutes of stirring at room temperature, the solutions were filtered through a coffee filter. The filtrate was collected and the volume of filtrate was measured. The concentration of oxycodone in the filtrate was determined by UPLC, in which the % extracted=100×(amount of oxycodone in filtrate)/(amount of oxycodone added).

Results

Results for % extraction Composition 1 and Oxycodone hydrochloride tablets 15 mg (commercially available) are provided in Table 2.

TABLE 2
Percent Extraction of Oxycodone

| | | Commercially |
|---|---|---|
| Solvent | Composition 1 (%) | available (%) |
| pH 1.2 Buffer (USP Hydrochloric | 47.4 | 84.0 |
| acid buffer) | | |
| pH 6.8 Phosphate Buffer | 57.9 | 81.7 |
| pH 10.0 Buffer (USP Alkaline | 4.4 | 10.7 |
| Borate Buffer) | | |
| 40% Ethanol | 40.7 | 79.5 |
| Ethanol | 75.6 | 83.9 |
| Acetone | 73.6 | 13.1 |
| 1% Acetic Acid | 70.4 | 84.2 |
| Water | 64.7 | 89.0 |
| Coco-Cola | 62.5 | 91.3 |
| Coco-Cola + 40% Ethanol | N/A | 75.2 |

### Example 3

### Filter Study

Materials and Methods

Composition 1 containing 15 mg of oxycodone hydrochloride was extracted.

Procedure: Composition 1 was crushed into a powder using a mortar and pestle. The powder of five tablets or ten tablets was transferred to a beaker. 5 ml or 10 ml of solvent (water, ethanol, or isopropyl alcohol (IPA)) was added to the beaker containing the powder of five tablets and ten tablets respectively. The mixture was stirred.

After stirring for 10 minutes at room temperature, the solution was filtered through either a 0.2 μm nylon filter, 0.4 μm nylon filter or a coffee filter. The amount of oxycodone extracted was determined by UPLC.

Results

The results are provided in Table 3.

TABLE 3
Oxycodone Extracted

| | 0.2 μm Nylon | 0.4 μm Nylon | Coffee Filter |
|---|---|---|---|
| Solvent | % oxycodone | | |
| H₂O | N/A | 76.8 | N/A |
| Ethanol | N/A | 88.7 | 92.1 |
| Isopropyl Alcohol | N/A | 11.8 | 13.2 |

### Example 4

### Syringe Needle Gauge Study

Materials and Methods

Composition 1 containing 15 mg of oxycodone hydrochloride was extracted.

Procedure: Each of the extraction solutions in Example 3 was passed through a syringe equipped with different gauge size needles. This study was performed to determine the needle gauge size needed to pass the solvent.

Results

Results are provided in Table 4.

TABLE 4
Syringe Needle gauge evaluation

| | | | | |
|---|---|---|---|---|
| Solvent | 27 G½ | 25 G⅝ | 22 G1 | 18 G1 |

5 tablets in 25 ml solvent (5 ml/tablet)

| | | | | |
|---|---|---|---|---|
| H₂O | Not Injectable | Not Injectable | Yes | Yes |
| Ethanol | Not Injectable | Not Injectable | Yes | Yes |
| IPA | Not Injectable | Not Injectable | Yes | Yes |

5 tablets in 15 ml solvent (3 ml/tablet)

| | | | | |
|---|---|---|---|---|
| H₂O | Not Injectable | Not Injectable | Yes | Yes |
| Ethanol | Not Injectable | Not Injectable | Yes | Yes |
| IPA | Not Injectable | Not Injectable | Yes | Yes |

### Example 5

### In Vitro Dissolution Studies Profile

Dissolution studies were performed using different media as described in table 5.

TABLE 5
Composition 1 Profile
Oxycodone HCl Tablets, 15 mg USP

Media

| | | | |
|---|---|---|---|
| Water | pH 1.2 | pH 4.5 | pH 6.8 |

| | |
|---|---|
| Time | Composition 1 |

| | | | | |
|---|---|---|---|---|
| 5 minutes | 39 | 23 | 29 | 71 |
| 10 minutes | 73 | 42 | 44 | 96 |
| 20 minutes | 92 | 68 | 71 | 100 |
| 30 minutes | 96 | 82 | 87 | 101 |
| 45 minutes | 98 | 93 | 99 | 101 |

500 ml, water, paddle at 50 RPM

TABLE 6
Oxycodone Hydrochloride tablets 15 mg immediate
release (commercially available) dissolution Profile
Oxycodone HCl Tablets, 15 mg USP

Media

| | | | |
|---|---|---|---|
| Water | pH 1.2 | pH 4.5 | pH 6.8 |

| | |
|---|---|
| Time | Commercially available |

| | | | | |
|---|---|---|---|---|
| 5 minutes | 63 | 33 | 45 | 45 |
| 10 minutes | 92 | 72 | 76 | 84 |
| 20 minutes | 96 | 96 | 95 | 96 |
| 30 minutes | 97 | 99 | 99 | 99 |
| 45 minutes | 98 | 100 | 100 | 100 |

500 ml, water, paddle at 50 RPM

These studies were conducted for comparative purposes.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A solid oral pharmaceutical composition comprising:
(a) an active agent or pharmaceutically acceptable salt thereof;
(b) a gelling agent in an amount of between about 0.7 to about 1.5% of the weight of the composition; and
(c) a channeling agent in an amount of at least about 40% of the weight of the composition.

2. The composition of claim 1, wherein the composition is an immediate release tablet.

3. The composition of claim 1, wherein the active agent is an opioid.

4. The composition of claim 3, wherein the opioid is oxycodone hydrochloride in an amount of between about 5mg and about 30 mg.

5. The composition of claim 4, wherein the opioid is oxycodone hydrochloride in an amount of about 15 mg.

6. The composition of claim 1, wherein the gelling agent is xanthan gum.

7. The composition of claim 6, wherein the xanthan gum is an amount of about 1.0% of the weight of the composition.

8. The composition of claim 1, wherein the channeling agent is crospovidone.

9. The composition of claim 6, wherein the crospovidone is in an amount about 53.3% of the weight of the composition.

10. The composition of claim 1 further comprising polyglycols.

11. The composition of claim 1 further comprising a cationic polymer.

12. The composition of claim 1 further comprising a lubricant.

13. The composition of claim 1 further comprising a film coating.

14. The composition of claim 10, wherein the polyglycols are polyethylene oxide with an average molecular weight of between about 900,000 daltons and about 7,000,000 daltons.

15. The composition of claim 11, wherein the cationic polymer is a methacrylic acid derivative with an amino alkyl methacrylate group.

16. The composition of claim 15, wherein the cationic polymer is poly(butyl methacrylate-co-(2-dimethylaminoethyl)methacrylate-co-methyl methacrylate) 1:2:1.

17. The composition of claim 12, wherein the lubricant is magnesium stearate.

18. The composition of claim 13, wherein the film coating comprises polyvinylalcohol.

19. A method of manufacturing the composition of claim 1 comprising:
(a) forming a mixture containing an active agent, a gelling agent and a channeling agent; and
(b) forming a solid dosage unit from the mixture.

20. A method of treating or preventing pain in a patient in need thereof comprising administering the composition of any of claims 1-18.
